(19) United States
(12) Patent Application Publication
(10) Pub. No.: US 2022/0296104 A1
(43) Pub. Date: Sep. 22, 2022

(54) PULSE METER AND CONTROL METHOD THEREFOR

(71) Applicant: Beijing Taiya Technology Co., Ltd., Beijing (CN)

(72) Inventors: Be XIE, Beijing (CN); Changhong YU, Beijing (CN)

(21) Appl. No.: 17/600,740

(22) PCT Filed: Apr. 2, 2020
(86) PCT No.: PCT/CN2020/082955

§ 371 Date: Apr. 28, 2022

(30) Foreign Application Priority Data
Apr. 4, 2019 (CN) 201910272281.9
Apr. 4, 2019 (CN) 201920454476.0

(51) Int. Cl. A61B 5/02; F04D 25/06

(52) U.S. Cl. CPC A61B 5/02; F04D 25/06; A61B 2562/0247

(57) ABSTRACT

Disclosed are a pulse meter and a control method therefor, aiming at solving the problem that a plurality of sensors in the existing pulse meter cannot fit well with the wrist surface. The pulse meter comprises a main body, a compression mechanism disposed on the main body, a plurality of pressure sensors (12) connected with the compression mechanism, and a controller (6) in communication connection with the compression mechanism and the pressure sensors (12). The compression mechanism comprises a compression airbag (21) and an air pump assembly connected with the compression airbag (21); the compression airbag (21), when inflated, can enable the plurality of presser sensors (12) to abut the wrist surface at approximately the same preset pressure. The described arrangement can enable the plurality of presser sensors (12) in the pulse meter to abut the wrist surface at approximately the same preset pressure, thereby enhancing the accuracy of collecting pulse manifestation, balancing the force on the wrist surface, and optimizing user experience.

### FIELD

The present disclosure relates to the technical field of pulse diagnosis, and specifically provides a pulse diagnosis instrument and a control method therefor.

### BACKGROUND

With the continuous development and progress of science and technology, more and more medical devices have been developed, out of which a pulse diagnosis instrument is typically provided with an airbag in its shell, and a sensor is arranged on the airbag. The airbag is inflated/deflated so as to be deformed so that the sensor comes into or out of contact with a wrist. The sensor collects pulse condition information when it contacts the wrist. By collecting the pulse condition information by the sensor, the accuracy of obtaining the pulse condition information is improved, thus preventing doctors from making incorrect judgments on the health condition of human body due to inaccurate pulse condition information, and avoiding the influence of the pulse diagnosis doctor's experience on the accuracy of pulse diagnosis, which greatly improves a diagnostic level and diagnostic efficiency of Chinese medicine.

In order to further improve the accuracy of the pulse condition information obtained by the pulse diagnosis instrument, an improved pulse diagnosis instrument appears on the market. A plurality of pressure sensors are arranged on the airbag of the pulse diagnosis instrument. When collecting the pulse condition information at the “Cun”, “Guan”, and “Chi” positions on the wrist, a plurality of pressure sensors are arranged at each of the three positions of “Cun”, “Guan” and “Chi” on the wrist to collect the pulse condition information, and the collected pulse condition information is processed to obtain more accurate pulse condition information, which improves the accuracy of pulse diagnosis. However, when the pressure sensor is pressed against the surface of the wrist after the airbag is inflated, since the surface of the airbag has a relatively large curvature, it is impossible for some of the plurality of pressure sensors to abut against the surface of the wrist or it is impossible for the plurality of pressure sensors to abut against the surface of the wrist at approximately the same pressure, which in turn affects the comprehensive and effective collection of pulse condition information by the plurality of pressure sensors.

Accordingly, there is a need in the art for a new technical solution to solve the above problem.

### SUMMARY

In order to solve the above problem in the prior art, that is, to solve the problem that a plurality of sensors of the existing pulse diagnosis instrument cannot fit well with the surface of the wrist, the present disclosure provides a pulse diagnosis instrument, which includes a body, a pressurizing mechanism arranged on the body, a plurality of pressure sensors connected to the pressurizing mechanism, and a controller communicatively connected to the pressurizing mechanism and the pressure sensors, in which the pressurizing mechanism includes a pressurized airbag and an air pump assembly connected to the pressurized airbag, and the pressurized airbag, after being inflated, enables the plurality of pressure sensors to abut against a surface of wrist at approximately the same set pressure.

In a preferred technical solution of the above pulse diagnosis instrument, the pressurized airbag includes a plurality of sub-airbags, and the plurality of sub-airbags are laid on one level so that the plurality of pressure sensors can abut against the surface of the wrist at the set pressure after the plurality of sub-airbags are inflated.

In a preferred technical solution of the above pulse diagnosis instrument, the plurality of sub-airbags communicate with each other, and/or the plurality of sub-airbags are arranged in an array on the same level.

In a preferred technical solution of the above pulse diagnosis instrument, the plurality of sub-airbags are arranged in an array on an annular level.

In a preferred technical solution of the above pulse diagnosis instrument, the pressurized airbag includes an elastic base and a plurality of air cavities formed in the base to communicate with each other, so that the plurality of pressure sensors abut against the surface of the wrist at approximately the same set pressure after the plurality of air cavities are inflated.

In a preferred technical solution of the above pulse diagnosis instrument, the pressurized airbag maintains an initial state when it is not inflated, and the shape of the pressurized airbag in the initial state matches the shape of the wrist.

In a preferred technical solution of the above pulse diagnosis instrument, the pressurized airbag includes two opposite pressed side walls and connection side walls connected to the two pressed side walls, and the pressed side walls and the connection side walls form a gas-containing cavity; in which a rigidity of the pressed side walls is greater than a rigidity of the connection side walls; and/or a thickness of the pressed side walls is larger than a thickness of the connection side walls.

In a preferred technical solution of the above pulse diagnosis instrument, the connection side wall includes a plurality of sub-side walls, and the plurality of sub-side walls are connected to each other to form a folded structure which is connected to the two pressed side walls respectively.

In a preferred technical solution of the above pulse diagnosis instrument, the air pump assembly includes an air pump for suctioning gas from the pressurized airbag.

In a preferred technical solution of the above pulse diagnosis instrument, a regulating valve is provided between the air pump assembly and the pressurized airbag, and the regulating valve is capable of adjusting a flow rate of the gas entering and exiting the pressurized airbag.

It can be understood by those skilled in the art that in the technical solutions of the present disclosure, the pressurized airbag in the pressurizing mechanism enables the plurality of pressure sensors to abut against the surface of the wrist at approximately the same set pressure after being inflated. For example, the pressurized airbag includes a plurality of sub-airbags, and the plurality of sub-airbags are laid on one level so that the plurality of pressure sensors abut against the surface of the wrist at approximately the same set pressure after the plurality of sub-airbags are inflated, or the pressurized airbag includes an elastic base and a plurality of air cavities formed in the base to communicate with each other, and after the plurality of air cavities are inflated, they enable the volume of the pressurized airbag to be increased, so that the plurality of pressure sensors abut against the surface of the wrist at approximately the same set pressure. Through such an arrangement, the plurality of pressure sensors in the pulse diagnosis instrument can abut against the surface of the wrist at approximately the same set pressure, which improves the accuracy of collecting pulse condition information, makes the forces on the surface of the wrist more uniform, and optimizes the user's experience in use, avoiding the situation in the existing pulse diagnosis instruments that some of the plurality of pressure sensors cannot abut against the surface of the wrist at the set pressure or cannot abut against the surface of the wrist, which would otherwise lead to the inability of some pressure sensors to collect effective pulse condition information or inaccurate pulse condition information after processing due to failing to collect the pulse condition information, thus resulting in inaccurate diagnosis or incorrect diagnosis of the health condition. It should be noted that the description of “the plurality of pressure sensors abut against the surface of the wrist at approximately the same set pressure” means that the pressures of the plurality of pressure sensors may be slightly different, that is, there may be a certain error range. For example, if the set pressure is 150g , the pressures of the plurality of pressure sensors are values between 140g and 160g , or if the set pressure is 80g , the pressures of the plurality of pressure sensors are values between75 g and 85 g, etc., which may be regarded as the situation of approximately the same pressure, and which does not have a substantive influence on the technical solutions.

In the preferred technical solutions of the present disclosure, the pressurized airbag includes a plurality of sub-airbags communicating with each other, and the plurality of sub-airbags are arranged in an array on one level. Through such an arrangement, not only the plurality of pressure sensors can abut against the surface of the wrist at approximately the same set pressure, but also the plurality of sub-airbags communicate with each other, so that the plurality of sub-airbags can be inflated with the aid of one air pump. As compared with a solution in which each of a plurality of sub-airbags independent from each other is equipped with one air pump for inflation, the structure of the present disclosure is relatively simple, the cost is relatively low, and the air pressures in the sub-airbags can be effectively kept consistent during the inflation process; when the plurality of pressure sensors abut against the wrist, the forces on the wrist are more even, which improves the comfort of the wrist during pulse diagnosis.

In addition, the present disclosure also provides a control method for a pulse diagnosis instrument, in which the pulse diagnosis instrument includes a body, a pressurizing mechanism arranged on the body, a plurality of pressure sensors connected to the pressurizing mechanism, and a controller communicatively connected to the pressurizing mechanism and the pressure sensors, the pressurizing mechanism includes a pressurized airbag and an air pump assembly connected to the pressurized airbag, and the control method includes the following steps: controlling the air pump assembly to inflate the pressurized airbag; controlling the pressure sensors to collect pulse condition information; and controlling the pressurized airbag to deflate; in which the inflation is performed according to a first set mode, and the deflation is performed according to a second set mode.

In a preferred technical solution of the above control method, the step that “the inflation is performed according to the first set mode” specifically includes: controlling the air pump assembly to inflate the pressurized airbag in stages.

In a preferred technical solution of the above control method, the step that “the inflation is performed according to the first set mode” specifically includes: controlling the air pump assembly to inflate the pressurized airbag at a gradually or stepwise decreasing inflation speed.

In a preferred technical solution of the above control method, the step that “the inflation is performed according to the first set mode” specifically includes: controlling the air pump assembly to inflate the pressurized airbag at a first set speed, and at the same time, controlling the pressurized airbag to deflate at a second set speed; in which the first set speed is larger than the second set speed.

In a preferred technical solution of the above control method, the step that “the deflation is performed according to the second set mode” specifically includes: controlling the pressurized airbag to deflate in stages.

In a preferred technical solution of the above control method, the step that “the deflation is performed according to the second set mode” specifically includes: controlling the pressurized airbag to deflate at a gradually or stepwise increasing deflation speed.

It can be understood by those skilled in the art that in the technical solutions of the present disclosure, the air pump assembly is controlled to inflate the pressurized airbag according to the first set mode during the inflation process, such as inflating in stages, and the pressurized airbag is controlled to deflate according to the second set mode during the deflation process, such as deflating in stages, which can make the pressure change in the pressurized airbag smooth, and avoid a fast change in the air pressure in the pressurized airbag during the inflation and deflation processes and discomfort of the wrist due to a fast change in the pressure on the wrist, thereby optimizing the user experience.

### BRIEF DESCRIPTION OF DRAWINGS

Preferred embodiments of the present disclosure will be described below with reference to the accompanying drawings, in which:

FIG. 1 is a schematic view showing a main structure of a pulse diagnosis instrument according to an embodiment of the present disclosure;

FIG. 2 is a schematic structural view of a pressurized airbag in FIG. 1;

FIG. 3 is a schematic structural view of a pressurized airbag of a pulse diagnosis instrument according to an embodiment of the present disclosure;

FIG. 4 is another schematic structural view of a pressurized airbag of a pulse diagnosis instrument according to an embodiment of the present disclosure;

FIG. 5 is a schematic sectional view of the pressurized airbag in FIG. 4;

FIG. 6 is another schematic structural view of a pressurized airbag of a pulse diagnosis instrument according to an embodiment of the present disclosure;

FIG. 7 is a schematic sectional view of the pressurized airbag in FIG. 6;

FIG. 8 is a diagram of the connection principle of a pressurizing mechanism and its related components in a pulse diagnosis instrument according to an embodiment of the present disclosure; and

FIG. 9 is a flowchart of a control method for a pulse diagnosis instrument according to an embodiment of the present disclosure.

### LIST OF REFERENCE SIGNS

11: substrate; 12: pressure sensor; 13: mounting plate; 14: elastic layer; 21: pressurized airbag; 211: pressed side wall; 212: connection side wall; 213: sub-airbag; 214: air pipe; 215: base; 216: air cavity; 217: air inlet; 218: air outlet; 22: air inlet and outlet pipe; 31: skin layer; 32: radial artery; 4: air pressure sensor; 51: inflating air pump; 52: vacuum pump; 6: controller; 7: regulating valve.

### DETAILED DESCRIPTION

Preferred embodiments of the present disclosure will be described below with reference to the accompanying drawings. It should be understood by those skilled in the art that these embodiments are only used to explain the technical principles of the present disclosure, and are not intended to limit the scope of protection of the present disclosure. For example, although the plurality of pressure sensors in the pulse diagnosis instrument of the present disclosure are indirectly connected to the pressurized airbag, those skilled in the art can make adjustment thereto as required so as to adapt to specific applications. For example, the plurality of pressure sensors may also be directly connected to the pressurized airbag, etc. Obviously, the adjusted technical solutions will still fall within the scope of protection of the present disclosure.

It should be noted that in the description of the present disclosure, terms indicating directional or positional relationships, such as “left”, “right”, “upper”, “lower”, “inner”, “outer” and the like, are based on the directional or positional relationships shown in the accompanying drawings. They are only used for ease of description, and do not indicate or imply that the device or element must have a specific orientation, or be constructed or operated in a specific orientation, and therefore they should not be considered as limitations to the present disclosure.

In addition, it should also be noted that in the description of the present disclosure, unless otherwise clearly specified and defined, terms “arrange” and “connect” should be understood in a broad sense; for example, the connection may be a fixed connection, or may also be a detachable connection, or an integral connection; it may be a mechanical connection, or an electrical connection; it may be a direct connection, or an indirect connection implemented through an intermediate medium, or it may be an internal communication between two elements. For those skilled in the art, the specific meaning of the above terms in the present disclosure can be understood according to specific situations.

In addition, in order to better illustrate the present disclosure, numerous specific details are given in the following specific embodiments. It should be understood by those skilled in the art that the present disclosure may also be implemented without certain specific details. In some embodiments, methods, means, elements, and circuits that are well known to those skilled in the art are not described in detail in order to highlight the spirit of the present disclosure.

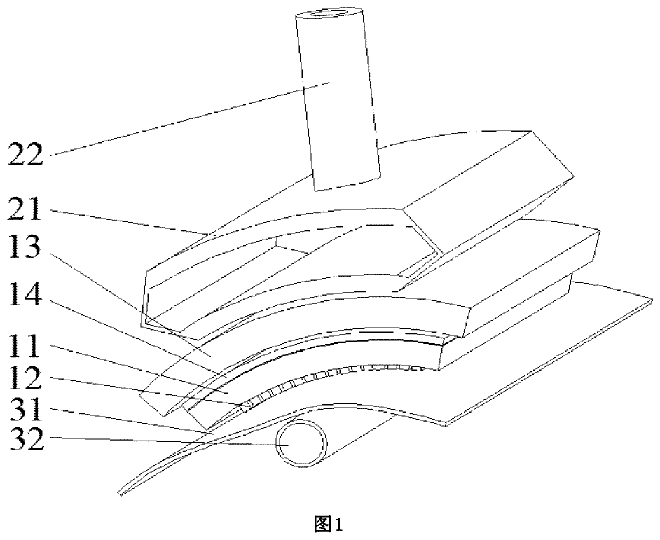

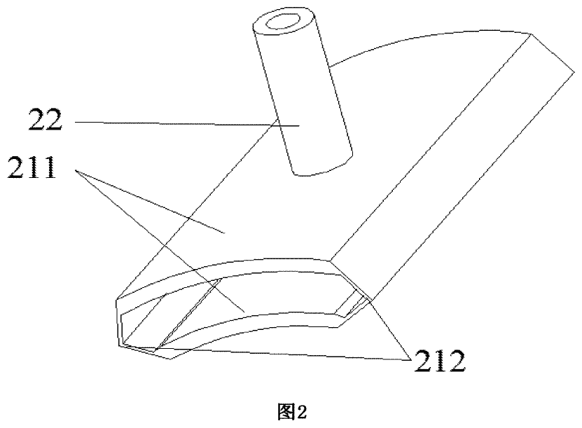

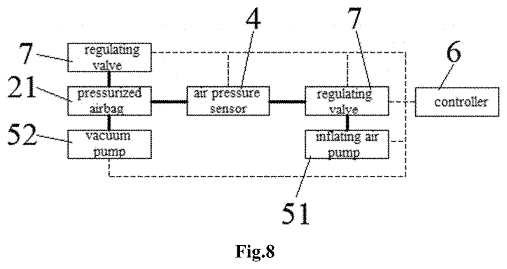

Reference is made to FIGS. 1, 2 and 8, in which FIG. 1 is a schematic view showing a main structure of a pulse diagnosis instrument according to an embodiment of the present disclosure, FIG. 2 is a schematic structural view of a pressurized airbag in FIG. 1, and FIG. 8 is a diagram of the connection principle of a pressurizing mechanism and its related components in a pulse diagnosis instrument according to an embodiment of the present disclosure.

As shown in FIGS. 1, 2 and 8 and in accordance with the orientation of FIG. 1, the pulse diagnosis instrument includes a body, such as a cylindrical shell (not shown in the figure). Inside the shell, a pressurizing mechanism, a plurality of pressure sensors 12 connected to the pressurizing mechanism, and a controller 6 communicatively connected to the pressurizing mechanism and the pressure sensors 12 are arranged. The pressurizing mechanism includes a pressurized airbag 21 and an air pump assembly connected to the pressurized airbag 21. Specifically, a mounting plate 13 is provided under the pressurized airbag 21, an elastic layer 14 is provided on a lower side of the mounting plate 13, and a substrate 11 is provided on a lower side of the elastic layer 14. The plurality of pressure sensors 12 are arranged in an array on a lower surface of the substrate 11. The pressurized airbag 21 includes two opposite pressed side walls 211 and connection side walls 212 connected to the two pressed side walls 211. The pressed side walls 211 and the connection side walls 212 form a gas-containing cavity. The pressed side walls 211 and the connection side walls 212 are made of the same material, such as polyethylene plastic, nylon, etc., and a thickness of the pressed side walls 211 is larger than a thickness of the connection side walls 212. For example, the thickness of the pressed side walls 211 is any value between 1 mm and 3 mm, and the thickness of the connection side walls 212 is any value between 0.05 mm and 0.5 mm. The upper pressed side wall 211 is provided with an air inlet and outlet pipe 22. The air pump assembly includes an inflating air pump 51 and a vacuum pump 52. The inflating air pump 51 and the vacuum pump 52 are connected to the air inlet and outlet pipe 22 through a three-way valve (not shown in the figure). An air pressure sensor 4 is provided in a pipeline between the inflating air pump 51 and the pressurized airbag 21. The controller 6 is communicatively connected with the pressure sensors 12, the air pressure sensor 4, the inflating air pump 51 and the vacuum pump 52, respectively. It should be noted that in order to facilitate illustrating the thicknesses of the pressed side walls 211 and the connection side walls 212, ends of the pressurized airbag 21 are not closed by the connection side walls 212, but this does not mean that the ends of the pressurized airbag in the pulse diagnosis instrument of the present disclosure are not closed.

During use, the controller 6 controls the inflating air pump 51 to start to inflate the pressurized airbag 21. In this process, the air pressure sensor 4 detects the air pressure value in the pipeline in real time (equivalent to the air pressure value in the pressurized airbag 21) and feeds it back to the controller 6 in real time. When the air pressure value in the pipeline reaches a set pressure value, the plurality of pressure sensors 12 abut against a skin layer 31 of the wrist at a set pressure, the controller 6 controls the inflating air pump 51 to stop, and at the same time controls the pressure sensors 12 to collect pulse condition information corresponding to “Cun”, “Guan” and “Chi” positions of a radial artery 32 on the skin layer 31. After the pulse condition information is collected by the pressure sensors 12, the controller 6 controls the vacuum pump 52 to pneumatically suction air from the pressurized airbag 21, so that the pressurized airbag 21 is restored to the initial state. For example, the pressurized airbag 21 may be in a vacuum state or a state in which there is a small amount of gas in the pressurized airbag 21, etc.

During the entire process of inflating the pressurized airbag 21, since the thickness of the pressed side walls 211 is larger than the thickness of the connection side walls 212, the shape of the pressed side walls 211 does not change much with the increase in the amount of inflation, and the shape of the connection side walls 212 changes greatly with the increase in the amount of inflation, so that the volume of the pressurized airbag 21 increases. Finally, under the pressure of the lower pressed side wall 211, the plurality of pressure sensors 12 are all subjected to the pressure transmitted from the substrate 11 so that they abut against the skin layer 31 of the wrist at approximately the same set pressure. Therefore, the plurality of pressure sensors 12 can all effectively collect pulse condition information at corresponding positions, thereby improving the accuracy of collecting the pulse condition information. In addition, the forces on the wrist are more even, which improves the comfort.

It can be understood by those skilled in the art that the pressed side walls 211 and the connection side walls 212 are made of the same material, such as polyethylene plastic, nylon, and the like. The body of the pulse diagnosis instrument being a cylindrical shell is only a specific embodiment, and those skilled in the art can adjust it as required so as to adapt to specific applications. For example, the body may be a ring-shaped structure composed of two semi-circular arch-shaped members, or may also be a ring-like structure formed by a flexible belt connected to a pressing mechanism (similar to the ring-like structure of a watch) or other suitable structures, as long as the body, the pressurizing mechanism and the wrist can cooperate to make the pressure sensors fit well with the “Cun”, “Guan” and “Chi” positions on the wrist. In addition, the thickness of the pressed side walls 211 being larger than the thickness of the connection side walls 212 is only a specific embodiment, and those skilled in the art can adjust it as required so as to adapt to specific applications. For example, the pressed side walls 211 and the connection side walls 212 are made of different materials; for example, the pressed side walls 211 are made of polyethylene, and the connection side walls 212 are made of nylon; or the pressed side walls 211 and the connection side walls 212 are made of other suitable different materials respectively, etc., and the thickness of the pressed side walls 211 is larger than the thickness of the connection side walls 212. In order that the main deformation occurs at the connection side walls 212 during the inflation process while the pressed side walls 211 basically maintain the shape thereof unchanged, the pressed side walls 211 may be set to have a greater rigidity than the connection side walls 212. For example, the pressed side walls 211 are made of resin and other materials, and the connection side walls 212 are made of rubber and other materials. The pressed side walls 211 and the connection side walls 212 have the same thickness, or the pressed side walls 211 may be set to have a greater rigidity and thickness than the connection side walls 212, or other suitable settings may be applied, etc. In addition, the air pump assembly including the vacuum pump 52 for suctioning air from the pressurized airbag 21 is only a preferred embodiment. Those skilled in the art may not provide the vacuum pump 52 as required, but a solenoid valve is provided instead, so that deflation of the pressurized airbag 21 can be achieved by controlling the solenoid valve to open. In addition, the pressurized airbag 21 being provided with the air inlet and outlet pipe 22 for inflation and deflation is only a specific embodiment, and those skilled in the art can adjust it so as to adapt to specific applications. For example, an air inlet pipe and an air outlet pipe may be provided on the pressurized airbag 21.

It can also be understood by those skilled in the art that the plurality of pressure sensors 12 being arranged in an array on the substrate 11, and the elastic layer 14 and the mounting plate 13 being provided between the substrate 11 and the pressurized airbag 21 so as to implement an indirect connection of the substrate 11 with the pressurized airbag 21 is only a specific embodiment, and those skilled in the art can adjust it as required so as to adapt to specific applications. For example, the plurality of pressure sensors 12 may be directly arranged on a lower side of the pressurized airbag 21.

With continued reference to FIG. 2, preferably, the connection side wall 212 includes two sub-side walls, and the two sub-side walls are connected to each other to form a “V”-shaped folded structure, which is connected to the two pressed side walls 211 respectively. Through such an arrangement, in the process of inflating and deflating the pressurized airbag 21, the folded structure can be deformed by folding and unfolding, so that the volume of the pressurized airbag 21 changes and the shape of the pressed side walls 211 maintains basically unchanged. One of the two pressed side walls 211 translates relative to the other to maintain the posture of the pressed side walls 211 basically unchanged, so that the plurality of pressure sensors 12 abut against the skin layer 31 of the wrist relatively accurately, and the plurality of pressure sensors 12 abut against the wrist at approximately the same set pressure, making the forces on the wrist more even.

It can be understood by those skilled in the art that the connection side wall 212 including two sub-side walls connected to each other to form a “V”-shaped folded structure is only a specific embodiment, and those skilled in the art can adjust it as required so as to adapt to specific applications. For example, the connection side wall 212 may include three, four or more sub-side walls, and the plurality of sub-side walls are connected to each other between each two to form a folded structure, or other suitable folding forms may be applied to form a folded structure, etc.

With continued reference to FIG. 8, preferably, the pressurized airbag 21 is also provided with a deflation port, and a regulating valve 7 is provided at the deflation port. In the process of deflating the pressurized airbag 21, the controller 6 controls an opening degree of the regulating valve 7 at the deflation port to adjust the deflation speed of the pressurized airbag 21, so that the pressurized airbag 21 can be deflated according to a set pressure curve, which further improves the comfort of the wrist. It should be noted that the opening degree of the regulating valve 7 can be adjusted between 0 and 100%. When the opening degree of the regulating valve 7 is 0, the regulating valve 7 is fully closed. When the opening degree of the regulating valve 7 is 100%, the regulating valve 7 is fully opened.

With continued reference to FIG. 8, preferably, a regulating valve 7 is provided on a pipeline between the inflating air pump 51 and the pressurized airbag 21. In the process of inflating the pressurized airbag 21, the controller 6 controls an opening degree of the regulating valve 7 between the inflating air pump 51 and the pressurized airbag 21 to adjust the inflation speed of the pressurized airbag 21, so that the pressurized airbag 21 is inflated according to a set pressure curve, which further improves the comfort of the wrist. It can be understood by those skilled in the art that the pressure curve may be an inclined straight line, a fold line or a smooth curve in the pressure-time coordinate system, etc.

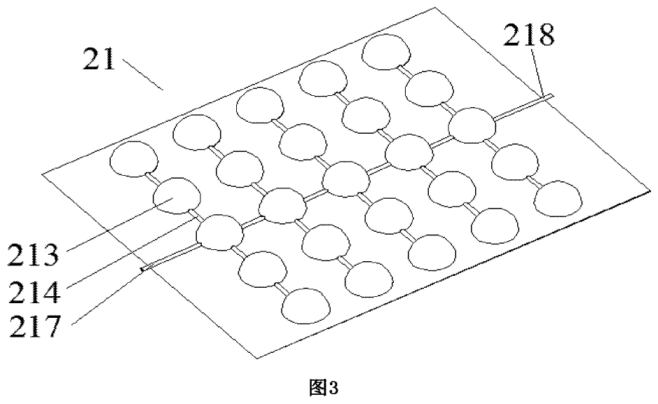

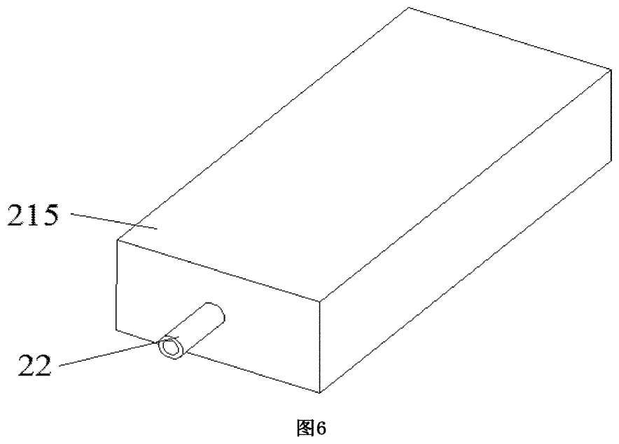

With continued reference to FIGS. 3 and 8, FIG. 3 is a schematic structural view of a pressurized airbag of a pulse diagnosis instrument according to an embodiment of the present disclosure. In another embodiment, as shown in FIGS. 3 and 6, the pressurized airbag 21 includes twenty-five (25) sub-airbags 213 arranged in a 5×5 array on one level, and the sub-airbags 213 communicate with each other through air pipes 214. The plurality of sub-airbags 213 are connected by a flexible material (such as plastic film, rubber film, etc.). One of the sub-airbags 213 is provided with an air inlet 217, and another sub-airbag 213 is provided with an air outlet 218. The inflating air pump 51 is connected to the air inlet 217 through a pipeline, and an air pressure sensor 4 and a regulating valve 7 are provided in a pipeline between the inflating air pump 51 and the air inlet 217. The air outlet 218 is respectively connected with a vacuum pump 52 and a regulating valve 7 through a three-way valve. The controller 6 can control the inflating air pump 51 to inflate the pressurized airbag 21 through the air inlet 217, and can control the regulating valve 7 between the inflating air pump 51 and the pressurized airbag 21 to change the opening degree thereof to adjust the inflation speed during the inflation process. The controller 6 can control any one of the vacuum pump 52 and the regulating valve 7 connected by the three-way valve to open, so as to implement the deflation by suctioning of the vacuum pump 52 or by controlling the deflation speed by the regulating valve 7 according to the set pressure curve for deflation.

Through such an arrangement, the volume of the plurality of sub-airbags 21 can be increased after the inflation, which applies a more uniform pressure to the plurality of pressure sensors 12 so that the plurality of pressure sensors 12 abut against the skin layer 31 of the wrist at approximately the same set pressure, which ensures that the plurality of pressure sensors 12 can well fit with the skin layer 31 of the wrist, improves the accuracy of collecting the pulse condition information, and improves the comfort of the wrist. The plurality of sub-airbags 213 are in communication with each other, so that the pressures in the plurality of sub-airbags 213 can maintain the same during inflation and deflation, making the forces on the wrist more uniform and improving the comfort. Moreover, as compared with a solution in which each of a plurality of sub-airbags independent from each other is equipped with one air pump for inflation, the structure of the present disclosure is relatively simple, and the cost is relatively low.

It can be understood by those skilled in the art that the pressurized airbag 21 including twenty-five (25) sub-airbags 213 arranged in a 5×5 array on one level is only a specific embodiment, and those skilled in the art can make adjustment thereto as required so as to adapt to specific applications. For example, the pressurized airbag 21 may include thirty (30), forty (40) or other numbers of sub-airbags 213, in which the thirty sub-airbags may be arranged in arrays of 10×3, 5×6, etc., and the forty sub-airbags may be arranged in arrays of 8×5, 10×4, etc. In addition, the plurality of sub-airbags 213 being in communication with each other is only a preferred embodiment, and those skilled in the art may also arrange the plurality of sub-airbags not to communicate with each other as required, and each of the sub-airbags is equipped with one inflating air pump for inflation.

Preferably, the plurality of sub-airbags 213 are arranged in an array on an annular level, and the plurality of sub-airbags 213 communicate with each other. Through such an arrangement, in the process of inflating and deflating the pressurized airbag 21, the entire round of the wrist can be subjected to uniform pressures, thereby further improving the comfort.

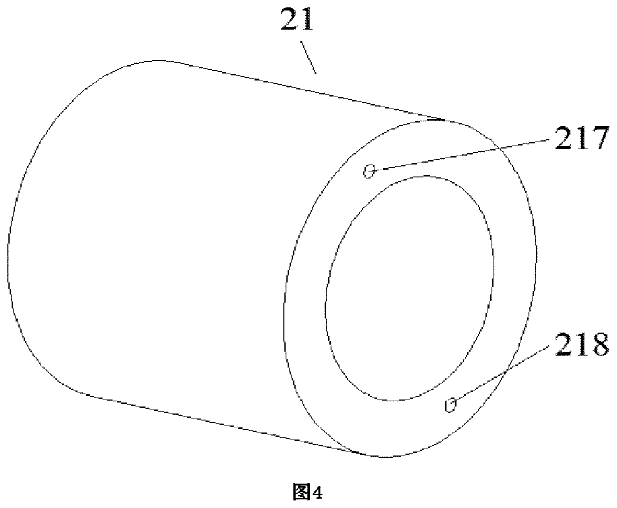

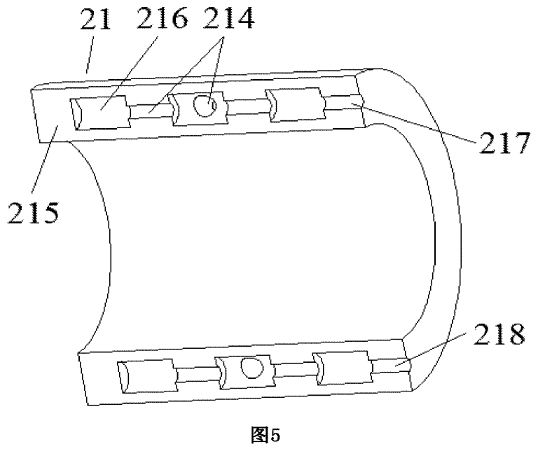

With reference to FIGS. 4 and 5, FIG. 4 is another schematic structural view of a pressurized airbag of a pulse diagnosis instrument according to an embodiment of the present disclosure, and FIG. 5 is a schematic sectional view of the pressurized airbag in FIG. 4. As shown in FIGS. 4 and 5, in another embodiment, the pressurized airbag 21 includes an annular base 215. The base 215 is made of a rubber material, and a plurality of air cavities 216 are formed in the base 215. The air cavities 216 communicate with each other through air pipes 214. The pressurized airbag 21 is also provided with an air inlet 217 and an air outlet 218. When the air cavities 216 are not inflated, the pressurized airbag 21 maintains the initial state, that is, a state in which the base 215 is not deformed. After the pressurized airbag 21 is inflated to the set pressure through the air inlet 217, the base 215 is deformed so that the volume of the pressurized airbag 21 is increased, and an inner ring of the annular pressurized airbag 21 is reduced so that the plurality of pressure sensors 12 abut against the skin layer 31 of the wrist. After the pulse condition information is collected by the pressure sensors 12, deflation is performed through the air outlet 218, such as by opening a solenoid valve connected at the air outlet 218. Under the action of the elastic force of the base 215 itself, the base 215 recovers from the deformation and discharges the gas in the air cavities 216 from the air outlet 218, so that the pressurized airbag 21 is restored to the initial state.

Through such an arrangement, the pressurized airbag 21 can maintain the initial state when it is not inflated, so as to facilitate the next pulse diagnosis operation. Relying on the elasticity of the base 215 itself, the gas in the air cavities 216 can be automatically discharged when the air outlet 218 is opened, and the pressurized airbag 21 can be automatically restored to the initial state, thereby making the pulse diagnosis operation more convenient. The base 215 has a ring shape, which matches the shape of the wrist, so that the base 215 can better fit the surface of the wrist after the pressurized airbag 21 is inflated and deformed, thereby improving the comfort.

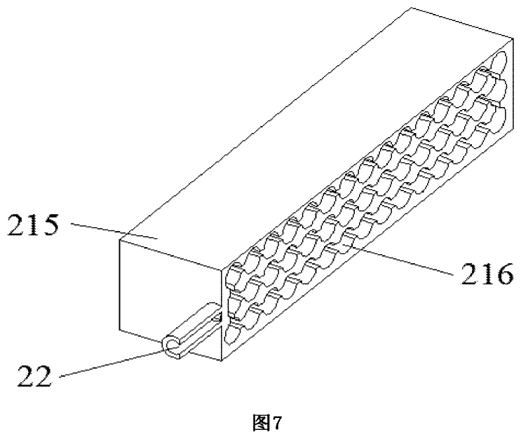

It can be understood by those skilled in the art that the base 215 being made of rubber is only a specific embodiment, and those skilled in the art can adjust it as required so as to adapt to specific applications. For example, the base 215 may also be made of silica gel or other suitable elastic materials. In addition, the base 215 having a ring-like structure is only a preferred embodiment, and those skilled in the art can adjust it as required so as to adapt to specific applications. For example, the base 215 may be saddle-shaped, cube-shaped, etc. The plurality of pressure sensors 12 are arranged on the side of the base 215 that is opposite to the “Cun”, “Guan” and “Chi” positions on the wrist. As shown in FIGS. 6 and 7, the base 215 is a cube provided with an air inlet and outlet pipe 22, and the base 215 is provided therein with a plurality of columnar air cavities 216. The plurality of columnar air cavities 216 are arranged in parallel in three rows with each row including a plurality of air cavities 216. The air cavities 216 adjacent to each other are in communication with each other, and the air cavities 216 at the ends of two adjacent rows are in communication with each other and with the air inlet and outlet pipe 22.

As can be seen from the above description, in the preferred technical solutions of the present disclosure, the pulse diagnosis instrument includes a body, a pressurizing mechanism provided on the body, a plurality of pressure sensors connected to the pressurizing mechanism, and a controller communicatively connected with the pressurizing mechanism and the pressure sensors, in which the pressurizing mechanism includes a pressurized airbag and an air pump assembly connected to the pressurized airbag, the pressurized airbag includes a plurality of sub-airbags laid on one level and communicating with each other so that the plurality of pressure sensors abut against the surface of the wrist at approximately the same set pressure after the sub-airbags are inflated; or the pressurized airbag includes a base and a plurality of air cavities formed in the base to communicate with each other, and after the plurality of air cavities are inflated, they enable the volume of the pressurized airbag to be increased, so that the plurality of pressure sensors abut against the surface of the wrist at approximately the same set pressure. Through such an arrangement, the plurality of pressure sensors in the pulse diagnosis instrument can abut against the surface of the wrist at approximately the same set pressure, which improves the accuracy of collecting pulse condition information, makes the forces on the surface of the wrist more uniform, and optimizes the user's experience in use, avoiding the situation in the existing pulse diagnosis instruments that some of the plurality of pressure sensors cannot abut against the surface of the wrist at approximately the same set pressure or cannot abut against the surface of the wrist, which would otherwise lead to the inability of some pressure sensors to collect effective pulse condition information or inaccurate pulse condition information after processing due to failing to collect the pulse condition information, thus resulting in inaccurate diagnosis or incorrect diagnosis of the health condition.

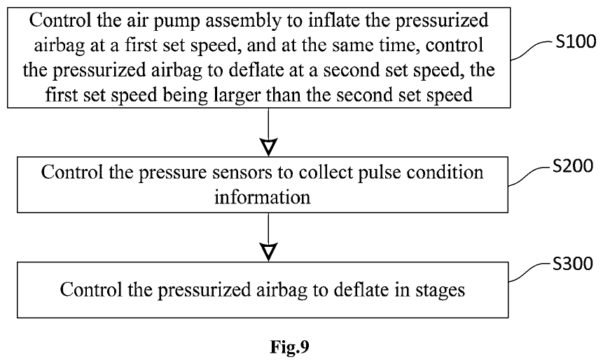

The control method for the pulse diagnosis instrument of the present disclosure will be introduced below with reference to FIGS. 3, 8 and 9.

As shown in FIGS. 3, 8 and 9, the pressurized airbag 21 of the pulse diagnosis instrument includes twenty-five (25) sub-airbags 213 arranged in a 5×5 array on one level, and the sub-airbags 213 communicate with each other through air pipes 214. The plurality of sub-airbags 213 are connected by a flexible material (such as plastic film, rubber film, etc.). One of the sub-airbags 213 is provided with an air inlet 217, and another sub-airbag 213 is provided with an air outlet 218. The inflating air pump 51 is connected to the air inlet 217 through a pipeline, and an air pressure sensor 4 and a regulating valve 7 are provided in a pipeline between the inflating air pump 51 and the air inlet 217. The air outlet 218 is respectively connected with a vacuum pump 52 and a regulating valve 7 through a three-way valve. The controller 6 is communicatively connected with the air pressure sensor 4, the inflating air pump 51, the vacuum pump 52, and the two regulating valves 7 respectively. The control method of the pulse diagnosis instrument of the present disclosure includes the following steps:

S100: controlling the air pump assembly to inflate the pressurized airbag at a first set speed, and at the same time, controlling the pressurized airbag to deflate at a second set speed; in which the first set speed is larger than the second set speed; S200: controlling the pressure sensors to collect pulse condition information; and S300: controlling the pressurized airbag to deflate in stages.

Specifically, the controller 6 controls the inflating air pump 51 to start; at the same time, the controller 6 controls the regulating valve 7 in the pipeline between the inflating air pump 51 and the air inlet 217 to adjust its opening degree to a first opening degree, such as 100%, controls the three-way valve so that the regulating valve 7 connected to the three-way valve communicates with the pressurized airbag 12, and controls the regulating valve 7 connected to the three-way valve to adjust its opening degree to a second opening degree, such as 20%. During this process, the air pressure sensor 4 detects the air pressure in the pressurized airbag 21 in real time. When the air pressure reaches the set pressure, the controller 6 controls the two regulating valves 7 to adjust their opening degrees to 0, so that the pressurized airbag 21 maintains the current air pressure. Then, the controller 6 controls the pressure sensors 12 to collect pulse condition information. After the pulse condition information is collected, the controller 6 controls the pressurized airbag 21 to deflate in stages. Specifically, the controller 6 controls the regulating valve 7 connected to the three-way valve to adjust its opening degree to 60%, then controls the regulating valve 7 to close after deflation for 2 seconds and maintains the pressure of the pressurized airbag for 2 seconds, then controls the regulating valve 7 to open to an opening degree of 60% for deflation for 2 seconds, and then controls the regulating valve 7 to close and maintains the pressure of the pressurized airbag for 2 seconds. This cycle is repeated until the gas in the pressurized airbag 21 is completely discharged.

That is, during the inflation process, deflation is also performed while inflating, and the inflation speed is larger than the deflation speed, so that an effective inflation amount of the pressurized airbag 21 changes relatively smoothly, thus avoiding a situation in which the pressurization speed in the pressurized airbag 21 is overly fast to cause the pressure on the wrist to change too much, and improving the comfort of the wrist. In addition, when the inflating air pump 51 is inflating the pressurized airbag 21, the load changes smoothly, which prolongs the service life of the inflating air pump 51 and reduces the use cost to a certain extent. In the deflation process, the deflation performed in stages can make the change in the pressure on the wrist relatively smooth, avoid the discomfort caused by a too fast decrease in the pressure on the wrist, and improve the comfort of the wrist during the deflation process. By deflating in stages, the wrist can be prevented from feeling uncomfortable during the deflation process due to a too fast change in the pressure on the wrist.

It can be understood by those skilled in the art that during the inflation process, the first opening degree being 100% and the second opening degree being 20% is only a specific embodiment, and those skilled in the art can adjust them as required. For example, the first opening degree is 90%, 80%, 85%, etc., and the second opening degree is 30%, 25%, 18%, etc., as long as the first opening degree is larger than the second opening degree. In addition, the deflation being performed in stages in a cycle of “deflating for 2 seconds—maintaining the pressure for 2 seconds—deflating for 2 seconds” is only an exemplary description, and those skilled in the art can adjust it as required. For example, the deflation may be performed in a cycle of “deflating for 0.5 seconds—maintaining the pressure for 0.5 seconds—deflating for 0.5 seconds” or “deflating for 0.5 seconds—maintaining the pressure for 0.3 seconds—deflating for 0.3 seconds” or at other time intervals.

In an alternative way of inflation, the pressurized airbag 21 can be inflated in stages. That is, after inflation for a first duration at a set inflation speed, the pressure is maintained for a second duration; then after inflation for a first duration at a set inflation speed, the pressure is maintained for a second duration; this cycle is repeated until the air pressure in the pressurized airbag 21 reaches a set value. The first durations may be the same or different. For example, the pressurized airbag 21 can be inflated in stages in a cycle of “inflating for 0.5 seconds—maintaining the pressure for 0.5 seconds—inflating for 0.5 seconds”, or “inflating for 0.8 seconds—maintaining the pressure for 0.2 seconds—inflating for 0.8 seconds”, etc. In this way, when the radial artery 32 is beating, it can avoid the inflation process of the pressurized airbag 21, and the comfort of the wrist can be improved.

In another alternative way of inflation, in the process of inflating the pressurized airbag 21, the inflation speed can be gradually reduced over time, so that the inflation is performed at a low inflation speed when the air pressure of the pressurized airbag 21 is close to a set value, thus making the air pressure change more smoothly and further improving the comfort. For example, before the pressurized airbag 21 contacts the wrist, the inflation speed gradually decreases from a large value during the inflation process, and the inflation speed continues to decrease when the pressurized airbag 21 contacts the wrist. Through such an arrangement, the pressurized airbag 21 is quickly inflated before it contacts the surface of the wrist, and is slowly inflated after the pressurized airbag 21 contacts the surface of the wrist, so that the comfort of the wrist during the inflation process is improved and a too long inflation time is avoided. It can be understood by those skilled in the art that the inflation speed can be reduced in stages.

In an alternative way of deflation, in the process of deflating the pressurized airbag 21, the deflation speed can be gradually increased over time, so that the deflation is performed at a low deflation speed when the air pressure of the pressurized airbag 21 is close to a set value, thus making the air pressure change more smoothly and further improving the comfort. It can be understood by those skilled in the art that the deflation speed can be increased in stages.

Hitherto, the technical solutions of the present disclosure have been described in conjunction with the preferred embodiments shown in the accompanying drawings, but it is easily understood by those skilled in the art that the scope of protection of the present disclosure is obviously not limited to these specific embodiments. Without departing from the principles of the present disclosure, those skilled in the art can make equivalent changes or replacements to relevant technical features, and all the technical solutions after these changes or replacements will fall within the scope of protection of the present disclosure.

1. A pulse diagnosis instrument, comprising a body, a pressurizing mechanism arranged on the body, a plurality of pressure sensors connected to the pressurizing mechanism, and a controller communicatively connected to the pressurizing mechanism and the pressure sensors, the pressurizing mechanism comprising a pressurized airbag and an air pump assembly connected to the pressurized airbag,
wherein the pressurized airbag, after being inflated, enables the plurality of pressure sensors to abut against a surface of wrist at approximately the same set pressure.

2. The pulse diagnosis instrument according to claim 1, wherein the pressurized airbag comprises a plurality of sub-airbags, and the plurality of sub-airbags are laid on one level so that the plurality of pressure sensors can abut against the surface of the wrist at the set pressure after the plurality of sub-airbags are inflated.

3. The pulse diagnosis instrument according to claim 2, wherein the plurality of sub-airbags communicate with each other, and/or the plurality of sub-airbags are arranged in an array on one level.

4. The pulse diagnosis instrument according to claim 3, wherein the plurality of sub-airbags are arranged in an array on an annular level.

5. The pulse diagnosis instrument according to claim 1, wherein the pressurized airbag comprises an elastic base and a plurality of air cavities formed in the base to communicate with each other, so that the plurality of pressure sensors abut against the surface of the wrist at approximately the same set pressure after the plurality of air cavities are inflated.

6. The pulse diagnosis instrument according to claim 5, wherein the pressurized airbag maintains an initial state when it is not inflated, and the shape of the pressurized airbag in the initial state matches the shape of the wrist.

7. The pulse diagnosis instrument according to claim 1, wherein the pressurized airbag comprises two opposite pressed side walls and connection side walls connected to the two pressed side walls, and the pressed side walls and the connection side walls form a gas-containing cavity;
wherein a rigidity of the pressed side walls is greater than a rigidity of the connection side walls; and/or a thickness of the pressed side walls is larger than a thickness of the connection side walls.

8. The pulse diagnosis instrument according to claim 7, wherein the connection side wall comprises a plurality of sub-side walls, and the plurality of sub-side walls are connected to each other to form a folded structure which is connected to the two pressed side walls respectively.

9. The pulse diagnosis instrument according to claim 1, wherein the air pump assembly comprises an air pump for suctioning gas from the pressurized airbag.

10. The pulse diagnosis instrument according to claim 9, wherein a regulating valve is provided between the air pump assembly and the pressurized airbag, and the regulating valve is capable of adjusting a flow rate of the gas entering and exiting the pressurized airbag.

11. A control method for a pulse diagnosis instrument, the pulse diagnosis instrument comprising a body, a pressurizing mechanism arranged on the body, a plurality of pressure sensors connected to the pressurizing mechanism, and a controller communicatively connected to the pressurizing mechanism and the pressure sensors, the pressurizing mechanism comprising a pressurized airbag and an air pump assembly connected to the pressurized airbag, wherein the control method comprises the following steps:
controlling the air pump assembly to inflate the pressurized airbag;
controlling the pressure sensors to collect pulse condition information; and
controlling the pressurized airbag to deflate;
wherein the inflation is performed according to a first set mode, and the deflation is performed according to a second set mode.

12. The control method according to claim 11, wherein the step that “the inflation is performed according to the first set mode” specifically comprises:
controlling the air pump assembly to inflate the pressurized airbag in stages.

13. The control method according to claim 11, wherein the step that “the inflation is performed according to the first set mode” specifically comprises:
controlling the air pump assembly to inflate the pressurized airbag at a gradually or stepwise decreasing inflation speed.

14. The control method according to claim 11, wherein the step that “the inflation is performed according to the first set mode” specifically comprises:
controlling the air pump assembly to inflate the pressurized airbag at a first set speed, and at the same time, controlling the pressurized airbag to deflate at a second set speed;
wherein the first set speed is larger than the second set speed.

15. The control method according to claim 11, wherein the step that “the deflation is performed according to the second set mode” specifically comprises:
controlling the pressurized airbag to deflate in stages.

16. The control method according to claim 11, wherein the step that “the deflation is performed according to the second set mode” specifically comprises:
controlling the pressurized airbag to deflate at a gradually or stepwise increasing deflation speed.
